(19) United States
(12) Patent Application Publication
(10) Pub. No.: US 2026/0314575 A1
(43) Pub. Date: Oct. 15, 2026

(54) ANIMAL MODEL OF TINNITUS INDUCED BY RESTRAINT STRESS

(71) Applicant: THE CATHOLIC UNIVERSITY OF KOREA INDUSTRY-ACADEMIC COOPERATION FOUNDATION, Seoul (KR)

(72) Inventors: Shi Nae PARK, Seoul (KR); Ye LIn KIM, Seoul (KR); Min Jung KIM, Seoul (KR)

(21) Appl. No.: 19/653,348

(22) Filed: Apr. 21, 2026

(30) Foreign Application Priority Data
Apr. 23, 2023 (KR) 10-2025-0053203

(51) Int. Cl. A01K 67/027

(52) U.S. Cl. CPC A01K 67/027; A01K 2207/35; A01K 2227/105; A01K 2267/03

(57) ABSTRACT

The present invention relates to a method of establishing a tinnitus animal model induced by restraint stress, and it is confirmed that when restraint stress is applied under specific conditions, it works as chronic stress to cause tinnitus as the altered neural activity in the auditory and limbic systems of the brain adapts. In addition, a mouse tinnitus model induced by chronic stress was established to observe the causal relationship between tinnitus and psychological symptoms, and thus can be useful in the method of establishing a tinnitus model using chronic stress developed from restraint stress.

### CROSS-REFERENCE TO RELATED APPLICATION

This application claims priority to and the benefit of Korean Patent Application No. 10-2025-0053203, filed on Apr. 23, 2025, the disclosure of which is incorporated herein by reference in its entirety.

### BACKGROUND

### 1. Field of the Invention

The present invention relates to a method of establishing a tinnitus animal model induced by restraint stress.

### 2. Discussion of Related Art

A disease model is used as a tool to experimentally recapitulate the pathological features of a specific disease, enabling the study of disease mechanisms and the development of therapeutic strategies. Disease models exist in various forms, including animal models, cell cultures, and organoid models. Such disease models enable researchers to experimentally analyze the etiology, progression, and therapeutic responses of diseases, providing critical preliminary data prior to drug development and clinical trials. These are also essential for evaluating novel therapeutic methods or preventive strategies.

Animal models refer to experimental animals designed to mimic specific biological characteristics to study human diseases. Studies using experimental animals can faithfully recapitulate human diseases, allowing real-time observation and analysis of pathological changes and therapeutic responses. In addition, animals with physiological features similar to humans enable researchers to evaluate long-term effects, toxicity, or drug metabolic pathways through experiments, and are used as a critical tool for ensuring safety before clinical trials. As such, animal models may deepen the understanding of diseases and provide an essential foundation for the development of novel therapies and drugs, thus being essentially used for drug discovery, investigation of disease pathogenesis, and validation of therapies.

Tinnitus is a common auditory disorder experienced by approximately 7 to 15% of the global population, and according to the WHO, hundreds of millions of people suffer from persistent tinnitus symptoms. Beyond mere discomfort, it may cause serious psychological and physical problems such as sleep disturbances, decreased concentration, anxiety, and depression, and some patients experience a significant decline in quality of life due to severe stress. Particularly, as society enters an aging phase, the number of tinnitus patients associated with age-related hearing loss is increasing, and the incidence is rising among the younger due to increased use of earphones.

In spite of this, few tinnitus models that can reflect high similarity and accuracy to date has been reported yet.

### PRIOR ART DOCUMENTS

### Patent Documents

(Korean Patent Publication) No. 10-2013-0127722

### DETAILED DESCRIPTION OF INVENTION

### Technical Problem

An object of the present invention is to provide a method of establishing a tinnitus animal model, comprising:
(S1) exposing animals to restraint stress (the animals are not humans); and
(S2) determining whether the restraint stress-exposed animals are tinnitus-positive, wherein the restraint stress satisfies any one or more conditions selected from the group consisting of:
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

Additionally, another object of the present invention is to provide a kit for establishing a tinnitus animal model, comprising a restraint stress-inducing apparatus and instructions,
wherein the restraint stress satisfies any one or more conditions selected from the group consisting of the following (the animals are not humans):
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

However, technical problems to be solved in the present invention are not limited to the above-described problems, and other problems which are not described herein will be fully understood by those of ordinary skill in the art from the following descriptions.

### Technical Solution

The present invention provides a method of establishing a tinnitus animal model, comprising:
(S1) exposing animals to restraint stress (the animals are not humans); and
(S2) determining whether the restraint stress-exposed animals are tinnitus-positive, wherein the restraint stress satisfies any one or more conditions selected from the group consisting of:

i) restraint density conditions consisting of complete restraint in which almost no movement is possible, partial restraint in which only movement of the head and torso is possible, and loose restraint in which voluntary movement is impossible but position changes are possible;

ii) restraint pressure conditions consisting of soft restraint applied with a flexible material, hard restraint applied with a rigid material, and variable-pressure restraint with adjustable pressure;

iii) restraint duration conditions consisting of single short-term restraint, repeated restraint lasting less than 14 days, and chronic restraint lasting 14 days or more; and
vi) restraint exposure conditions consisting of enclosed restraint and open restraint.

In one embodiment of the present invention, the restraint stress may be characterized by complete restraint, soft restraint, repeated restraint, and open restraint conditions, but is not limited thereto.

In one embodiment of the present invention, in the step (S1), the restraint stress may be applied for 30 to 180 minutes for 5 to 13 days, but is not limited thereto.

In one embodiment of the present invention, in the step (S2), the determining of whether the animals are tinnitus-positive may be performed by determining the animals as tinnitus-positive when the mean gap/no-gap (GN) ratio calculated by the following formula after exposure to restraint stress exceeds the 95% confidence interval of the GN ratio before exposure to restraint stress in a gap pre-pulse inhibition of acoustic startle (GPIAS) analysis method, but is not limited thereto.

[GN ratio]=(mean amplitude of gap-trials/mean amplitude of no-gap-trials)×100

In one embodiment of the present invention, the animal may be any one selected from the group consisting of a mouse and a rat, but is not limited thereto.

In one embodiment of the present invention, the tinnitus animal may be characterized by maintaining hearing function, but is not limited thereto.

In one embodiment of the present invention, the tinnitus animal may have an increased plasma corticosterone level, but is not limited thereto.

In one embodiment of the present invention, the tinnitus animal may be characterized in any one or more regions selected from the group consisting of Cornu Ammonis 1 (CA1), Cornu Ammonis 2 (CA2), and Cornu Ammonis 3 (CA3) of the hippocampus; the amygdala, and the inferior colliculus, by exhibiting any one or more selected from the group consisting of the following, but is not limited thereto: a significantly decreased gamma-aminobutyric acid (GABA) expression level, or a significantly increased expression level of any one or more selected from the group consisting of NMDA receptor subunit 1 (nr1) and NMDA Receptor Subunit 2B (n2b).

The present invention provides a kit for establishing a tinnitus animal model, comprising a restraint stress-inducing apparatus and instructions, wherein the restraint stress satisfies any one or more conditions selected from the group consisting of the following (the animals are not humans):
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

In one embodiment of the present invention, the instructions may teach any one of the above, but is not limited thereto.

### ADVANTAGEOUS EFFECTS

According to the method of establishing a tinnitus animal model induced by restraint stress, it was confirmed that when restraint stress is applied under specific conditions, it acts as chronic stress to cause tinnitus as the altered neural activity in the auditory and limbic systems of the brain adapts. In addition, a rat tinnitus model induced by chronic stress was established to observe the causal relationship between tinnitus and psychological symptoms, and thus the method of establishing a tinnitus model using chronic stress induced by restraint stress can be usefully applied.

### DESCRIPTION OF DRAWINGS

FIG. 1 shows an experimental design for establishing a tinnitus model induced by restraint stress.

FIGS. 2A and 2B show the responses at 8 kHz, 16 kHz, and 20 kHz in a control group (CG), a salicylate-induced tinnitus group (STG), a restraint stress-induced tinnitus group (RTG), and a restraint stress-induced non-tinnitus group (RNTG).

FIG. 2A shows results indicating that no significant ABR threshold differences were observed among all groups.

FIG. 2B shows that no differences in responses from low to high frequencies of f2 were observed in distortion product otoacoustic emissions (DPOAE) among all groups.

(Error bars represent standard error of the mean, analysis of variance, and Scheffe post-hoc values; CG=control group; STG=salicylate sodium-injected tinnitus group; RTG=restraint stress-induced tinnitus group; RNTG=restraint stress-induced non-tinnitus group.)

FIG. 2C shows the analysis results of the gap response of gap pre-pulse inhibition of acoustic startle (GPIAS) to tone bursts at 8 kHz, 16 kHz, and 20 kHz. (#vs STG, @ vs RTG; error bars represent standard error of the mean, analysis of variance, and Scheffe post-hoc values; *p<0.05, **p<0.01, ***p<0.001).

FIGS. 3A to 3C show the results of analyzing behavioral (depression and anxiety) and physiological changes after chronic restraint stress.

FIG. 3A shows the result of the EPM test for analyzing the level of anxiety, indicating that the tinnitus group spent significantly less time in the open arms. FIG. 3B shows the result of the FST test for analyzing the level of depression, indicating that immobility time was significantly increased in the tinnitus group compared to the control group. FIG. 3C shows the analysis results of body weight and plasma corticosterone level as physiological changes.

FIGS. 4A to 4F show the immunohistochemical analysis results of brain tissues in the inferior colliculus, amygdala, and hippocampal regions (images were captured at 80× magnification, with a scale bar representing 200 μm included at the lower right of each figure (magnification 80) (scale bar=200 μm)).

FIGS. 4A and 4C show the differential expression of GABA, NR1, and N2B receptors in CA2, CA3, and CA1 of the hippocampus in the tinnitus rat model (magnification 80).

FIGS. 4B and 4D show the quantitative analysis results of GABA, NR1, and N2B receptor expression in the hippocampal regions of FIGS. 4A and 4C, comparing quantitatively the expression of GABA, NR1, and N2B receptors in CA2, CA3, and CA1 of the hippocampus. (Error bars represent standard error of the mean, analysis of variance, and Scheffe post-hoc values; *p<0.05, **p<0.01, ***p<0.001. GABA=γ-aminobutyric acid receptor α1; NR1=N-methyl-D-aspartate receptor 1; N2B=N-methyl-D-aspartate receptor subunit 2b.)

FIGS. 4E and 4F show the results of immunohistochemical studies using antibodies against GABA Rα1, NMDAR1, and NMDAR2B in amygdala tissues.

FIG. 4E shows representative images of γ-aminobutyric acid (GABA), N-methyl-D-aspartate receptor (NR)1, and NR2B expression in the amygdala of each group. (All images were captured at 200× magnification, with a scale bar representing 100 μm included at the lower right of each figure (magnification 200) (scale bar=100 μm)).

FIG. 4F shows a graph quantifying the results of FIG. 4E. (Error bars represent standard error of the mean, analysis of variance, and Scheffe post-hoc values; *p<0.05, **p<0.01, ***p<0.001.)

FIGS. 4G and 4H show the results of immunohistochemical studies using antibodies against GABAARα1, NMDAR1, and NMDAR2B in the inferior colliculus (IC).

FIG. 4G shows representative images of γ-aminobutyric acid (GABAARα1), N-methyl-D-aspartate receptor (NR)1, and NR2B expression levels in the IC of each group. (All images were captured at 200×0 magnification, with a scale bar representing 100 μm included at the lower right of each figure (magnification 200) (scale bar=100 μm)).

FIG. 4H shows a graph quantifying the results of FIG. 4G. (Error bars represent standard error of the mean, analysis of variance, and Scheffe post-hoc values; *p<0.05, **p<0.01, ***p<0.001).

FIG. 5 shows a schematic that illustrates the mechanism of tinnitus development caused by stress.

### Best Mode

The present invention discloses a method of establishing a tinnitus animal model by applying restraint stress to animals. In vivo, restraint stress does not induce direct physical pain compared to other stress-inducing methods and is similar to psychological stress in humans, and thus it is commonly used as an emotional stress model. Accordingly, in the present invention, tinnitus occurred when restraint stress was applied to mice under specific conditions, and it was confirmed that tinnitus was successfully induced by verifying the reduction of markers in various regions including the hippocampus, thereby completing the present invention.

The present invention provides a method of establishing a tinnitus animal model, comprising:
(S1) exposing animals to restraint stress (the animals are not humans); and
(S2) determining whether the restraint stress-exposed animals are tinnitus-positive, wherein the restraint stress satisfies any one or more conditions selected from the group consisting of:
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

In the present invention, “restraint stress” may be defined as stress applied by using a device, space, or the like that can restrict the movement of a subject, and restraint stress may be classified into restraint density conditions, restraint pressure conditions, restraint duration conditions, and restraint exposure conditions.

In the present invention, “restraint density” may mean the degree of spatial allowance in restraint stress, and may be classified into complete restraint, partial restraint, and loose restraint.

Here, “complete restraint” may mean restraint in a narrow space where the animal can hardly move; “partial restraint” may mean restraint in a space where only the head or torso is fixed and only slight movement of the limbs is possible; and “loose restraint” may mean restraint in which some space remains such that voluntary movement is impossible but slight positional change is possible.

In the present invention, “restraint pressure” may mean the degree of contact force applied to the skin or skeleton of the animal under restraint stress, and may be classified into soft restraint, hard restraint, and adjustable-pressure restraint.

Here, “soft restraint” may mean a form in which the physical pressure applied to the animal is low because it is made of a flexible material, and may be, for example, when using restraint devices made of rubber, soft plastic, or cloth, but is not limited thereto. “Hard restraint” may mean a form in which the body is fixed with a rigid material, having little elasticity so that internal movement is almost impossible, and may be, for example, when using restraint devices made of acrylic or hard plastic, but is not limited thereto. “Adjustable-pressure restraint” may be a form in which pressure can be adjusted by having an air-injection pad, Velcro tightening, or rotary adjustment mechanism inside.

In the present invention, “restraint duration” may mean the condition of duration of restraint stress, and may be classified into acute restraint, repeated restraint, and chronic restraint.

Here, “acute restraint” may mean restraint applied only once or in a single session per day, generally within 30 minutes to 2 hours. “Repeated restraint” may mean restraint repeatedly applied for a certain time over several days, lasting less than 14 days, and may be, for example, restraint applied for 1 to 3 hours per day for 2 to less than 14 days. “Chronic restraint” may mean restraint lasting for 2 weeks or more, for example, long-term restraint applied for 2 to 6 hours per day for 14 days or more.

In the present invention, “restraint exposure” may mean whether ventilation or stimulus exposure is allowed during restraint, and may be classified into enclosed and open types.

Here, “enclosed restraint” may mean that the internal airflow is restricted, which may allow high-temperature conditions or accumulation of carbon dioxide, and may be applied, for example, using a spherical tube-type restraint device, but is not limited thereto. “Open restraint” may mean that airflow is allowed during restraint and that exposure to temporal or olfactory stimuli is possible, and may be applied, for example, using a grid-type restraint device or a ventilated cone device, but is not limited thereto.

In one embodiment of the present invention, when restraint stress was applied for 2 hours per day for a total of 10 days using a Decapicone characterized by a cone-shaped form made of vinyl as the restraint device, it was confirmed that a tinnitus-positive response occurred in the mouse model. The Decapicone used in the present invention may be a form extremely similar to the cone-shaped vinyl Decapicone of Braintree Scientific. Therefore, in one embodiment of the present invention, the restraint stress may be characterized by complete restraint, soft restraint, repeated restraint, and open restraint conditions, but is not limited thereto.

In the present invention, the restraint device may be any restraint device generally used in the art that can apply restraint stress characterized by complete restraint, soft restraint, repeated restraint, and open restraint conditions, and in one embodiment of the present invention, a Decapicone was used, but is not limited thereto. Restraint devices generally used according to the type of animal may be employed.

In one embodiment of the present invention, in the step (S1), the restraint stress may be applied for 30 to 180 minutes for 5 to 13 days, but is not limited thereto.

For example, in the present invention, the restraint stress may be applied under the condition of 5 to 13 days, 5 to 12 days, 5 to 11 days, 5 to 10 days, 6 to 13 days, 6 to 12 days, 6 to 11 days, 6 to 10 days, 7 to 13 days, 7 to 12 days, 7 to 11 days, 7 to 10 days, 8 to 13 days, 8 to 12 days, 8 to 11 days, 8 to 10 days, 9 to 13 days, 9 to 12 days, 9 to 11 days, 9 to 10 days, or 10 days, but is not limited thereto.

For example, in the present invention, the restraint stress may be 30 to 160 minutes, 30 to 150 minutes, 30 to 140 minutes, 30 to 130 minutes, 30 to 120 minutes, 60 to 180 minutes, 60 to 160 minutes, 60 to 150 minutes, 60 to 140 minutes, 60 to 130 minutes, 60 to 120 minutes, 80 to 180 minutes, 80 to 160 minutes, 80 to 150 minutes, 80 to 140 minutes, 80 to 130 minutes, 80 to 120 minutes, 100 to 180 minutes, 100 to 160 minutes, 100 to 150 minutes, 100 to 140 minutes, 100 to 130 minutes, 100 to 120 minutes, 110 to 180 minutes, 110 to 160 minutes, 110 to 150 minutes, 110 to 140 minutes, 110 to 130 minutes, 110 to 120 minutes, or 120 minutes, but is not limited thereto.

In one embodiment of the present invention, in the step (S2), the determining of whether the animals are tinnitus-positive may be performed by determining the animals as tinnitus-positive when the mean gap/no-gap (GN) ratio calculated by the following formula after exposure to restraint stress exceeds the 95% confidence interval of the GN ratio before exposure to restraint stress in a gap pre-pulse inhibition of acoustic startle (GPIAS) analysis method, but is not limited thereto.

[GN ratio]=(mean amplitude of gap-trials/mean amplitude of no-gap-trials)×100

In the present invention, GN was used to evaluate tinnitus perception by the GPIAS method. In tinnitus, the concepts of gap and no-gap are related to the neural signal transmission of the animal. Gap tinnitus may mean a phenomenon in which neural signals are interrupted at a specific frequency band, while no-gap tinnitus may mean a state in which the signals are continuously transmitted without interruption. Gap tinnitus may occur due to auditory fatigue, neural damage, or specific noise environments, and generally may occur when neurons in the auditory pathway are damaged or function incompletely.

In the present invention, the “gap ratio” may mean the proportion of time in which neural signals are interrupted by a specific stimulus, and may be used to evaluate auditory function. The higher the gap ratio, the more impaired the auditory processing ability is considered to be, and it may serve as an indicator for measuring neurological damage or tinnitus severity. The gap phenomenon is associated with noise-induced hearing loss, aging, and neurological diseases, and serves as a criterion for evaluating auditory processing ability in animal experiments. To confirm this, a gap detection test is used, which is employed to analyze auditory function in animals. When the gap ratio increases in a specific frequency band, sensitivity to sound may decrease. Therefore, by analyzing the gap ratio, the extent of damage and the possibility of recovery of the auditory nervous system may be evaluated. Accordingly, the concepts of gap and no-gap, and the gap ratio may have important significance in auditory research.

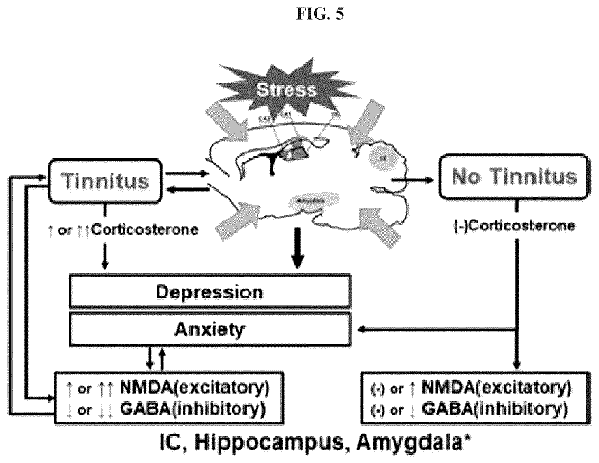

In the present invention, it was shown that chronic restraint stress may induce tinnitus in 64.3% of rats, confirming that stress may be involved in the occurrence of tinnitus. FIG. 5 illustrates the mechanism of tinnitus development caused by stress.

In one embodiment of the present invention, the animal may be any one selected from the group consisting of a mouse and a rat, but is not limited thereto.

In one embodiment of the present invention, the tinnitus animal may be characterized by maintaining hearing function, but is not limited thereto.

In one embodiment of the present invention, the tinnitus animal may have an increased plasma corticosterone level, but is not limited thereto.

In one embodiment of the present invention, the tinnitus animal may be characterized in any one or more regions selected from the group consisting of Cornu Ammonis 1 (CA1), Cornu Ammonis 2 (CA2), and Cornu Ammonis 3 (CA3) of the hippocampus; the amygdala; and the inferior colliculus, by exhibiting any one or more selected from the group consisting of the following, but is not limited thereto:
a significantly decreased gamma-aminobutyric acid (GABA) expression level, or a significantly increased expression level of any one or more selected from the group consisting of NMDA receptor subunit 1 (nr1) and NMDA receptor subunit 2b (n2b).

A tinnitus model produced by drugs is an important tool for tinnitus research, but several limitations may exist. A drug-induced model may often fail to fully reproduce the specific causes or mechanisms by which tinnitus occurs, and the temporary auditory damage or inflammatory responses induced by the drug may differ from the persistent characteristics of tinnitus. In addition, tinnitus induced by drugs may differ from the complex pathological characteristics of tinnitus observed in humans, thereby limiting the direct applicability of results from animal models to humans. Furthermore, side effects caused by drug use or individual variability may also reduce the consistency of research results. For these reasons, there are limitations to understanding the overall mechanisms of tinnitus using only drug models, and an approach combining various models is required. However, in the present invention, a tinnitus animal model was successfully established using restraint stress without drugs, thereby producing a tinnitus animal model in which tinnitus was induced similarly to the drug-induced tinnitus animal model and tinnitus disease, while eliminating the disadvantages associated with drug use.

The present invention provides a kit for establishing a tinnitus animal model, comprising a restraint stress-inducing apparatus and instructions,
wherein the restraint stress satisfies any one or more conditions selected from the group consisting of the following (the animals are not humans):
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

In one embodiment of the present invention, the instructions may teach any one of the above, but is not limited thereto.

In the present invention, the term “kit” means a tool that enables the establishment of a tinnitus animal model by including the restraint stress-inducing apparatus of the present invention. The kit of the present invention may further include other components, compositions, solutions, or devices conventionally required for storage and handling of the above materials. Specifically, each component may be applied one or more times without limitation in the number of applications, there is no limitation on the order in which each material is applied, and each material may be applied simultaneously or sequentially.

In the present invention, the kit may include a container, instructions, and the like. The container may serve to package the above substance, and may also serve to store and secure it. The material of the container may take the form of, for example, a bottle, tub, sachet, envelope, tube, or ampoule, and may be formed partially or entirely from plastic, glass, paper, foil, wax, or the like. The container may be equipped with a cap that is initially a part of the container or is completely or partially detachable and attachable to the container by mechanical, adhesive, or other means, and may also be equipped with a stopper that allows access to the contents with a syringe needle. The kit may include an external package, and the external package may include instructions for use of the components.

Hereinafter, preferred examples are presented to help understand the present invention. However, the following examples are provided only to help understand the present invention more easily, and the contents of the present invention are not limited by the following examples.

### EXAMPLES

### Animals

Male Sprague-Dawley rats (7 weeks old, 285 to 310 g) were purchased from the Orient Bio Animal Center (Seongnam, South Korea). The experiments strictly complied with ethical guidelines, including the Laboratory Animal Welfare Act, the Guide for the Care and Use of Laboratory Animals, and Institutional Animal Care and Use Committee protocols at the School of Medicine (Approval No. 2021-0205-01).

### Experimental Condition Setting

A total of 44 rats were categorized into four groups. A control group (CG) (n=10), and a sodium salicylate-injected (350 mg/kg, Sigma-Aldrich, Saint Louis, MO) tinnitus group (hereinafter referred to as a salicylate-induced tinnitus group (STG)) (n=6) as a positive control were intraperitoneally administered to the rats for 10 consecutive days.

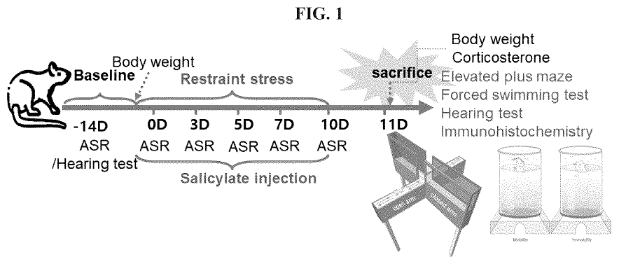

Twenty-eight rats were initially exposed to stress, anticipating variability in their responses to chronic stress. After GPIAS tests, these rats were divided into two experimental groups of a restraint stress-induced tinnitus group (RTG) (n=18) and a restraint stress-induced non-tinnitus group (RNTG) (n=10), which was determined based on whether tinnitus developed after restraint stress exposure. Restraint stress was applied to the rats using a cone-shaped DecapiCone restrainer made of vinyl (FIG. 1), while only their noses were exposed to the outside, for 2 hours daily (10 am to 12 pm) for 10 days.

### Auditory Brainstem Response (ABR) and Distortion Product Otoacoustic Emissions (DPOAE)

Hearing function was evaluated using ABR and DPOAE before and after restraint stress. The rats were anesthetized using Rompun^{®}(0.4 mL/kg) and Zoletil^{®}(0.6 mL/kg). ABR thresholds and DPOAE levels were measured and compared between the groups using a SmartEP device.

Gap response of Pre-Pulse Inhibition Acoustic Startle (GPIAS)

In the present invention, tinnitus perception was assessed by the gap response of pre-pulse inhibition acoustic startle (hereinafter, GPIAS) method using the equipment and methods which have been described in the previous literature. According to the GPIAS protocol, each rat was subjected to a combination of 15 no-gap trials and 15 gap trials, which were randomly conducted. The trials were executed at intervals of 17 to 23 seconds after the 2 min adaptation period for each rat. The gap trials included 60 dB SPL continuous narrowband noise (NBN) centered at 8, 16, and 20 kHz, and a 50 ms silent gap ending 100 ms before the startling stimulus (110 dB, white noise).

Pre-stress GPIAS assessment established a baseline, excluding the initial 3-day adaptation results from analysis. The software automatically calculated the average of the respective trials of gaps and no-gaps, and calculated a gap/no-gap (GN) ratio: (mean amplitude of gap-trials/mean amplitude of no-gap-trials)×100. A higher GN ratio indicates a larger defect of GPIAS. In the present invention, the GN ratio was calculated from the daily amplitude result. A significant increase in GN ratio indicates tinnitus as compared with the control (CG), and this was analyzed using a t-test for statistical significance. The GPIAS test was carried out immediately, and 3, 5, 7, and 10 days after stress exposure.

### Behavioral Tests for Anxiety and Depression Conditions

An anxiety level was measured using an elevated plus maze (EPM) test. The EPM apparatus was composed of a pair of open arms (50×10×70 cm) and a pair of closed arms of the same size with 40 cm high walls. The rat was placed in the central square of the apparatus facing the open arms and allowed to explore the maze for 5 minutes. The numbers of open-arm entries and closed-arm entries, and time spent in the open arms were recorded.

FST was used to evaluate depression behavior. FST was performed in a cylindrical tank (height: 60 cm, diameter: 30 cm) containing water at a height of 40 cm (25 °C±1 °C) for 2 days. On the first day, the rat underwent a 15-minute swim session. After one day (24 hours), the rat was subjected to a 5-minute session under the same swim conditions. During the session, immobility time was recorded.

### Assessment of Body Weight and Plasma Corticosterone Level

A stress level was assessed by monitoring a body weight and evaluating a plasma corticosterone concentration. The blood was readily collected from the cardiac region, and immediately subjected to centrifugation (Centrifuge UNION 32R PLUS; Hanil Science Industrial Co., Ltd., Incheon, Korea) at 13,000 rpm and 4 °C for 15 minutes to isolate the plasma. Corticosterone was determined using a corticosterone competitive enzyme-linked immunosorbent assay (ELISA) kit (Abcam, Cambridge, UK) in accordance with the manufacturer's instructions, and measured at 450 nm using an ELISA reader.

### Immunohistochemical Staining of Inferior Colliculus, Hippocampus, and Amygdala

Immunohistochemical staining was performed by the following method. On the day of sacrifice, the rats were perfused transcardially with phosphate-buffered saline (PBS), followed by perfusion with a 4% paraformaldehyde (PFA) solution in PBS at room temperature. The brain was extracted, and tissue samples were fixed overnight in 4% PFA. Subsequently, the samples were left in a 30% sucrose solution at 4 °C until they sank, and then 30 μm thick coronal sections were obtained using a cryostat microtome (Leica, Wetzlar, Germany) and stored at −20 °C. For immunohistochemical staining, the brain sections were washed with PBS, briefly incubated in 3% H₂O₂ for 10 minutes, washed twice with PBS, and incubated in 1.5% BSA in PBS for 1 hour. Subsequently, the sections were treated with anti-GABAARα1 (#06-868; Millipore, CA, USA; 1:1000), anti-NMDAR1 (#32-0500; Thermo Fisher Scientific, MA, USA; 1:200), and anti-NMDAR 2B (#ab 65783 ; Abcam, Cambridge, UK; 1:200), and left in 0.5% BSA in PBS overnight at room temperature.

After washing twice with 0.5% BSA, the sections were exposed to biotinylated rat or rabbit antibodies (#ba-1100; Vector Laboratories, Burlingham, California; 500:1) in 0.5% BSA at room temperature for 2 hours. After washing twice with 0.5% BSA, the brain sections were treated with an avidin-biotin complex (ABC) reagent (#pk-4002, Vector Laboratories, Burlingham, California; 500:1) for 2 hours. The sections were developed using a diaminobenzidine peroxidase substrate kit (#sk-4100, Vector Laboratories, Burlingham, California), mounted on slides, air-dried, and cover-slipped to be used in microscopic observation. Immunohistochemical images were captured using an AX70 microscope (Olympus Corporation), and staining intensity was calculated with ImageJ in accordance with standard protocols.

### Data Analysis

Data were analyzed using SPSS 20.0 (IBM Corp., Armonk, NY, USA). Statistical significance was determined using the Student t-test or ANOVA with Scheffe post-hoc test.

### Example 1. Confirmation of Effect on Tinnitus Development and Hearing Function in Restraint Stress-Induced Tinnitus model

### Example 1-1. Assessment of Hearing Function

The hearing function of tinnitus rat models established by a method of establishing a tinnitus model induced by restraint stress of the present invention was assessed.

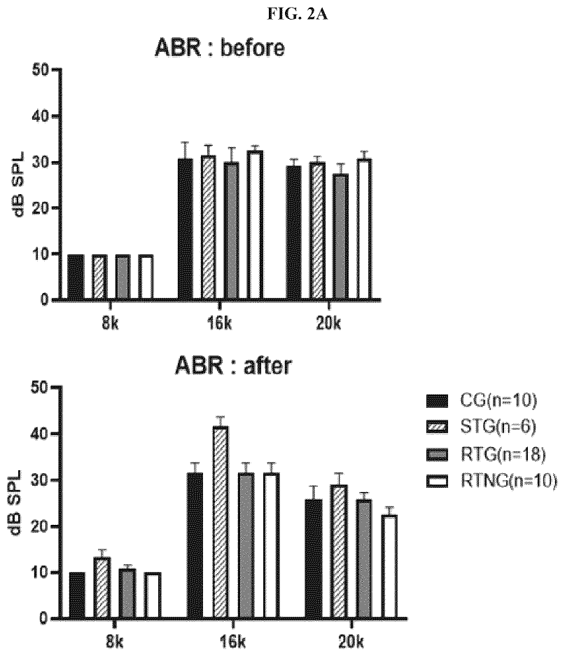

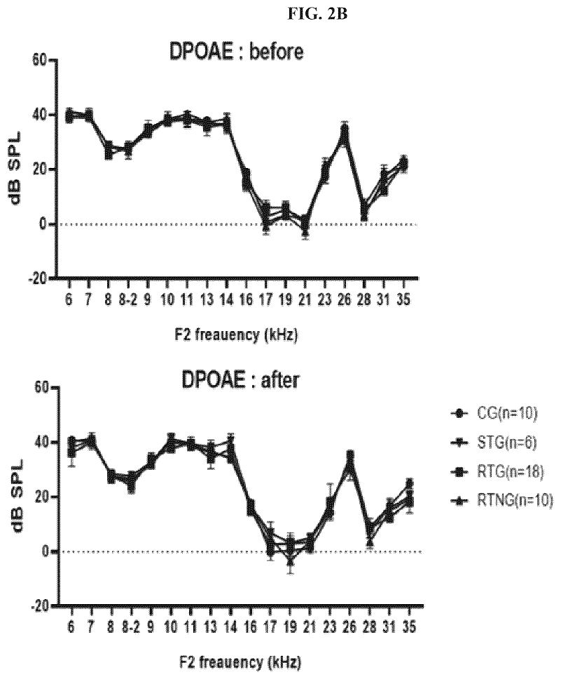

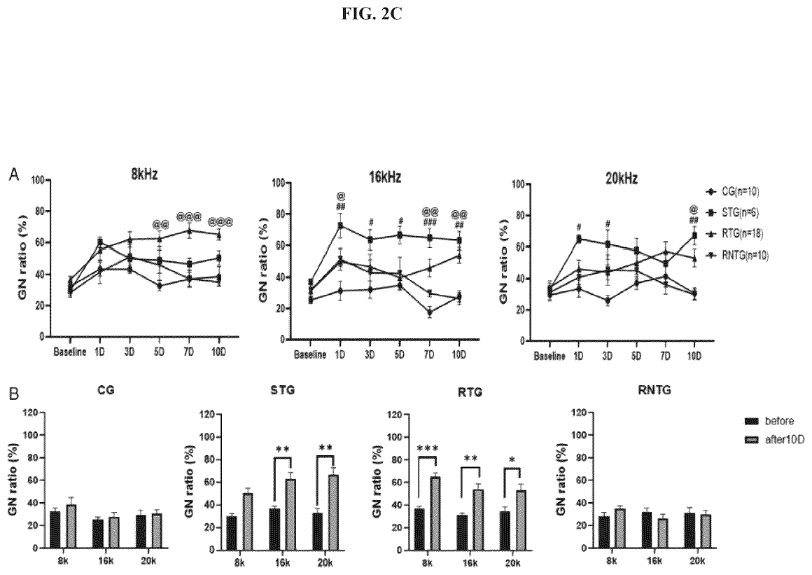

The results indicate that before intervention, ABR thresholds for 8, 16, and 20 kHz frequencies were relatively consistent throughout all groups. After intervention, the salicylate-induced tinnitus group (STG) showed increased thresholds at 16 kHz (31.67 dB SPL) and 20 kHz (30 dB SPL), whereas the restraint stress-induced tinnitus group (RTG) and the restraint stress-induced non-tinnitus group (RNTG) showed minimal changes (FIG. 2A).

DPOAE levels were similar before intervention. After intervention, STG showed DPOAE levels at 16 kHz and 20 kHz that had decreased below those before intervention (FIG. 2B).

Hearing tests showed no significant level deviation in all groups.

### Example 1-2. GPIAS Measurement

First, for GPIAS measurement, rats with tinnitus (hereinafter, tinnitus-positive) were identified. The criteria of “tinnitus-positive” were defined as a GN ratio greater than the 95% confidence interval of the mean GN ratio before exposure to restraint stress of the group. At each time point, the number of tinnitus-positive rats was counted within the group

As a result, the GN ratio in each group per frequency depending on the exposure to restraint stress was confirmed as in A of FIG. 2C. In addition, 10 days after restraint stress exposure, the GN ratio in each group per frequency was confirmed as in B of FIG. 2C.

Accordingly, the GN ratios before restraint stress exposure had no significant difference between the groups FIG. 2C).

First (A of FIG. 2C), the GN ratios which were measured daily showed increased values in STG and RTG at 8 kHz, especially on day 5 after restraint stress exposure or drug administration, the ratios increased (p<0.05, p<0.01). On the first day of the stress exposure, RNTG tended to increase at 16 kHz compared to CG, which was not statistically significant, but STG and RTG showed significant increases compared to CG on the first day (p<0.05). The GN ratio at 16 kHz significantly increased again in RTG on day 10 (p<0.01). Both STG and RTG showed significantly increased values compared to CG on day 10 (p<0.05 and 0.01, respectively).

Meanwhile (B of FIG. 2C), after restraint stress exposure for 10 days, RTG and STG showed significantly increased GN ratios compared to CG, and even on day 10 after exposure, RTG and STG showed significantly higher GN ratios than those before exposure.

According to the above-mentioned experimental results, similar GN ratio patterns in STG and RTG indicate that stress and drug-induced tinnitus exhibit similar biological characteristics. Although some ABR thresholds in STG are higher than 40 dB, GPIAS measurement at a 60 dB background sound level is likely unaffected due to normal hearing levels at other frequencies.

### Example 2. Analysis of Psychological Changes in Restraint Stress-Induced Tinnitus Model

### Example 2-1. Analysis of Depression and Anxiety Levels

For psychological change analysis in restraint stress-induced tinnitus models, depression and anxiety levels were analyzed. Here, for anxiety level analysis, an EPM test was performed, and for depression level analysis, an FST test was performed.

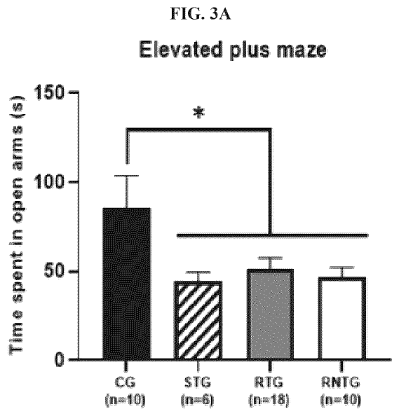

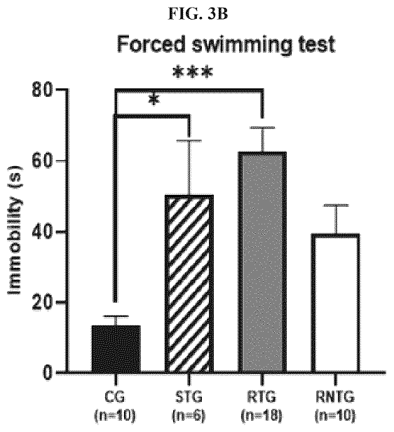

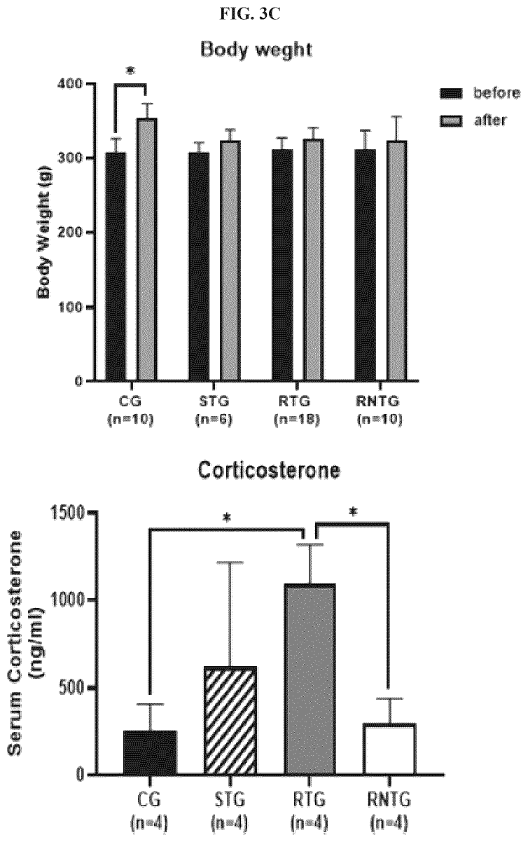

The results of the EPM test showed significant differences in the rate of open-arm entries between two groups. All of RTG, STG, and RNTG showed significantly lower rates of open-arm entries compared to CG, but there were no significant differences among STG, RTG, and RTNG (FIG. 3A). The FST test revealed that the immobility time in CG was significantly shorter than those in RTG and STG. On the other hand, there was no significant difference in immobility time between CG and RNTG (FIG. 3B).

In this example, anxiety levels measured by EPM indicated a significant increase after chronic stress exposure, and STG showed an increased anxiety level, indicating that tinnitus has a potential contribution to anxiety. Particularly, RTG showed a higher anxiety level than other groups, further indicating a relevance between tinnitus and anxiety after stress exposure. However, the increased anxiety level in RNTG also emphasizes the importance of recognition of chronic stress as an independent factor contributing to anxiety.

In addition, in relation to depression behavior changes, FST, RTG and STG showed a significantly longer immobility time than RNTG and CG, indicating potential relevance of depression and chronic stress to tinnitus. Among all groups, RTG showed the most serious depressive condition, confirming that stress and tinnitus have a greater influence on depression.

### Example 2-2. Assessment of Body Weight and Plasma Corticosterone Level

For psychological change analysis in the restraint stress-induced tinnitus models, body weights and plasma corticosterone levels were assessed.

The results (FIG. 3C) showed no significant difference in body weight between any two groups before stress started, but a significant increase in body weight was observed only in CG after day 10 (p<0.05). In addition, plasma corticosterone levels were statistically significantly increased only in RTG (p<0.05).

According to the above-mentioned experimental results, it was investigated that tinnitus was associated with increased neural activity of the auditory system. In addition, the increased neural activity may be caused by an imbalance of neurotransmitters, indicating that fluctuations in chemical transmitters can disrupt brain function and cause depression and anxiety, in addition to causing tinnitus.

### Example 3. Confirmation of Marker Changes Due to Restraint Stress and Tinnitus

To investigate the effects of stress and tinnitus on GABAARα1, NMDAR1 and NMDAR2B expression in various regions of the hippocampus, amygdala, and brain in the experimental groups, immunohistochemical staining was conducted.

First, the staining analysis results in the hippocampus are as follows.

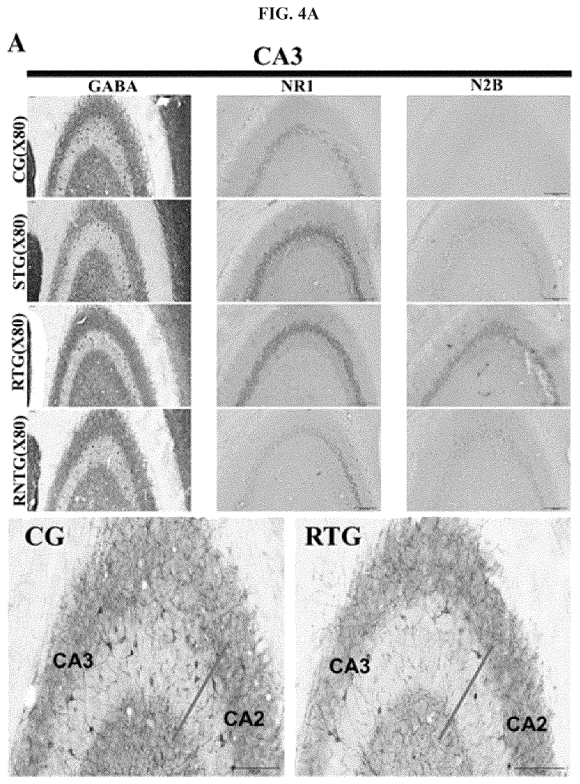

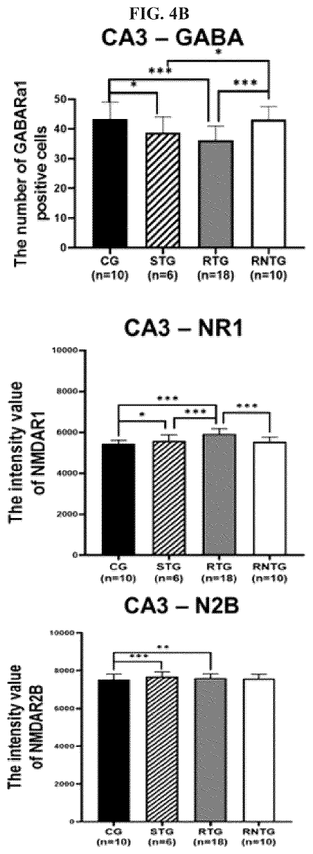

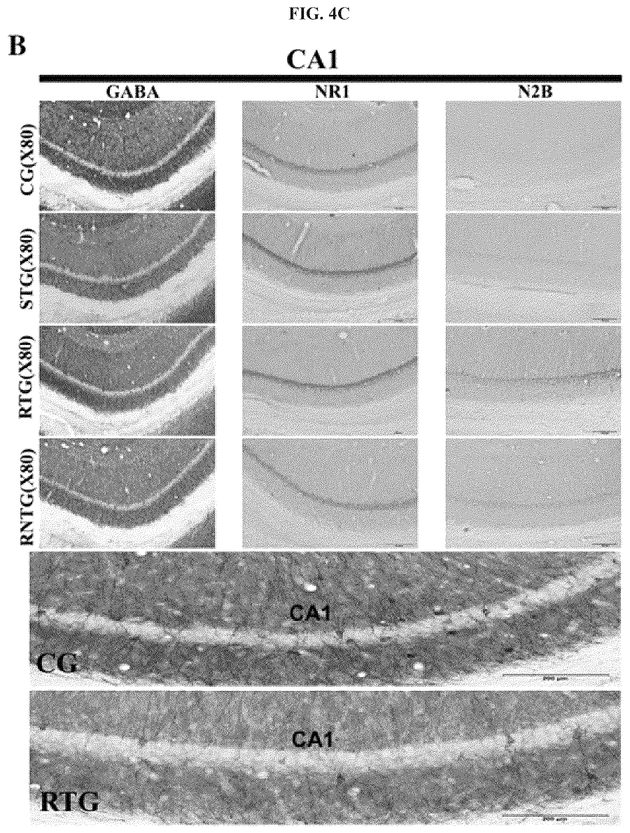

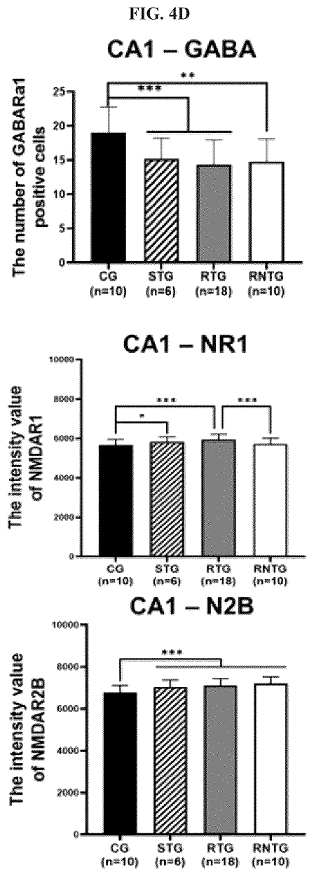

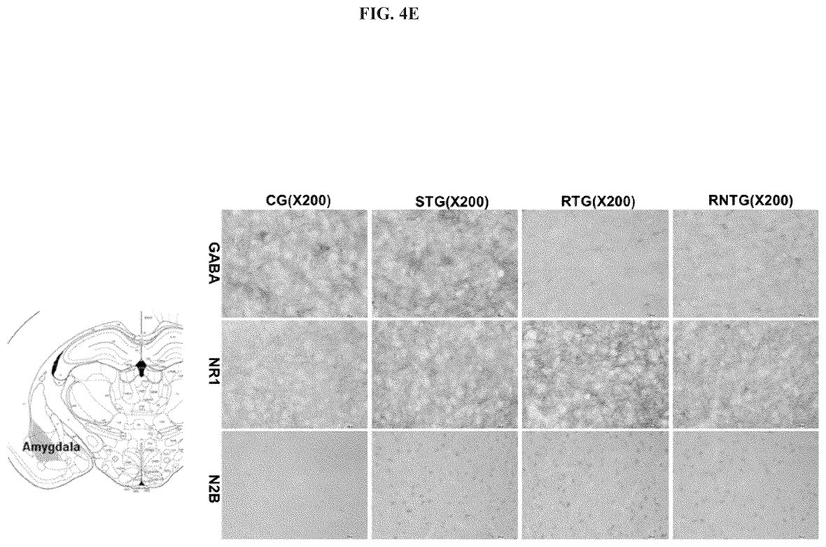

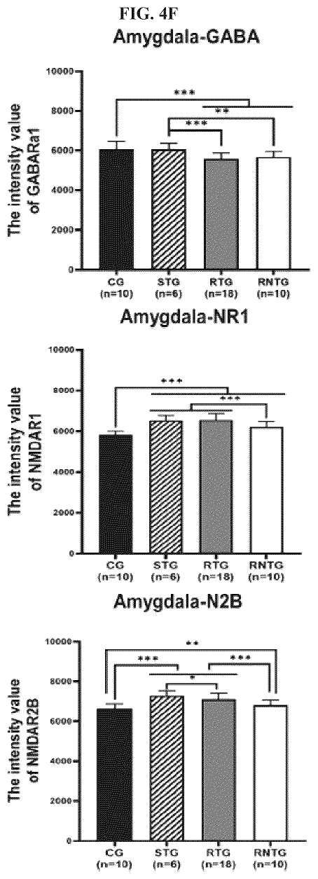

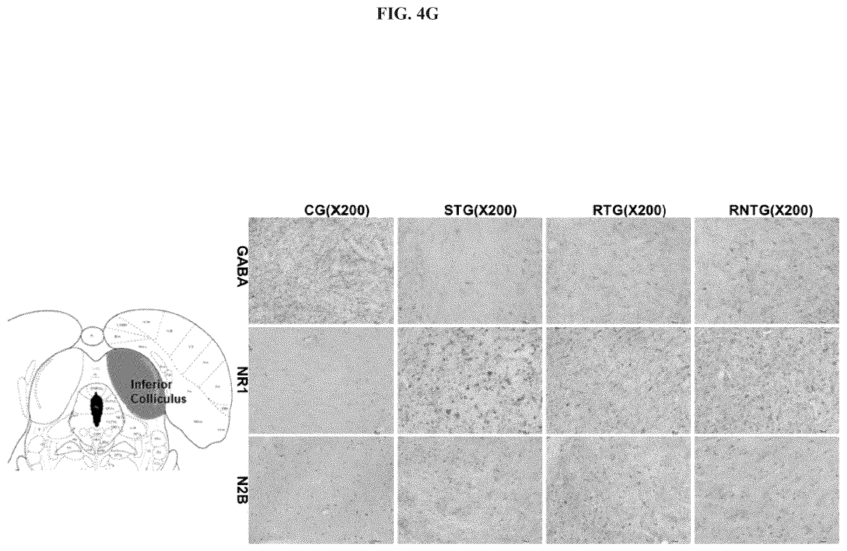

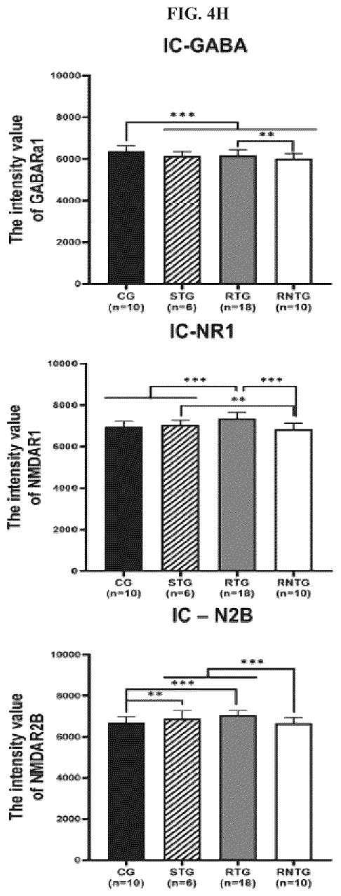

The results for CA2 and CA3 of the hippocampus in RTG and STG showed that GABAARα1 expression was significantly reduced compared to those of CG and RNTG (p<0.001 and p<0.05, respectively) (FIGS. 4A and 4B). This shows that, in the groups in which tinnitus was induced by restraint stress or drug stress, GABA was significantly reduced, and in CA2 and CA3, NR1 and N2B expression was significantly increased.

On the other hand, the numbers of GABAARα1-and N2B-positive cells in CA1 of the hippocampus were significantly lower in CG than other groups (p<0.01 in RNTG relative to CG, p<0.001 in STG and RTG relative to CG, and FIGS. 4C and 4D). Particularly, the tinnitus groups showed significantly increased NR1 expression in CA1.

The above-mentioned results indicate that there are significant differences in receptor expression among the control group (CG) and the tinnitus model groups (STG and RTG), and other treatment groups (CG and RTG).

The results from the amygdala and the inferior colliculus (hereinafter, IC) are as follows.

The GABARα1 intensity levels were significantly reduced in the RTG rats, compared to those of the other groups, but the NMDAR1 and NMDAR2B intensity levels in the amygdala were significantly increased in STG and RTG rats compared to CG and RNTG rats (FIG. 4F). In addition, in the IC of the CG rats, GABAARα1 expression was significantly higher than those of all of the other groups (p<0.001). It was confirmed that the NMDAR1 and NMDAR2B intensities in the IC were significantly increased in the RTG rats compared to those of all of the other groups (p <0.001) (FIG. 4H).

Specifically, RTG showed significantly lower GABAARα1 expression compared to CG and STG (p<0.001, FIG. 4F). In the IC, the GABAARα1 expression level was highest in CG, significantly surpassing all other groups (p<0.001, FIG. 4H).

In the hippocampus, NMDAR1 expression in CA2, CA3, and CA1 was considerably higher in RTG than those in CG (p<0.001, FIGS. 4B and 4D). Likewise, STG showed increased levels in both regions (p<0.05, FIGS. 4B and 4D). In addition, in the amygdala, STG and RTG showed significant upregulation of NMDAR1 compared to CG and RNTG (p<0.001, FIG. 4F).

In the IC, the improved expression of NMDAR1 was significantly higher in RTG, compared to those of the other groups (p<0.001, FIG. 4H), and STG also showed significantly higher expression than RNTG (p<0.01, FIG. 4H).

Relating to NMDAR2B, the expression levels in the IC were significantly increased in STG and RTG, compared to that of RNTG (p<0.001, FIG. 4H), and the expression level in RNTG was also increased compared to that of CG (p<0.01, FIG. 4H). This pattern was also reflected in CA2 and CA3 of the hippocampus, and STG and RTG experienced significant increases in NMDAR2B expression in contrast to CG (p<0.01 for CG vs. RTG, and p<0.001 for CG vs. STG, FIG. 4B). In the amygdala, NMDAR2B expression was similarly increased in STG and RTG, but not in CG and RNTG (p<0.001, FIG. 4F). STG showed the highest expression compared to RTG (p<0.05, FIG. 4F).

In this example, a significant relevance between the limbic system, including the amygdala and hippocampus, and the tinnitus brain network was observed, which supports a tinnitus-related brain network. According to the experimental results, GABAARα1 expression was significantly reduced in the hippocampal CA2 and CA3 of RTG and STG, indicating stress-related reduction in inhibitory neurotransmission in the limbic system. In addition, quite a contrast was observed in GABAARα1, NMDAR1, and NMDAR2B receptor expression in these regions, indicating that the reorganization of neural circuits that can be involved in tinnitus development is related to auditory impairments and tinnitus. The hippocampus and IC in STG and RTG also showed increased NMDAR1 and NMDAR2B levels, pointing out a more active excitatory neurotransmission related to tinnitus generation. This corresponds to the relevance between tinnitus and the NMDA receptors. The amygdala of the RTG rats also showed the same expression pattern, whereas the IC showed reduced GABA R expression, and had a significant difference in NMDAR and NMDAR2B expression between groups.

It should be understood by those of ordinary skill in the art that the above description of the present invention is exemplary, and the exemplary embodiments disclosed herein can be easily modified into other specific forms without departing from the technical spirit or essential features of the present invention. Therefore, the exemplary embodiments described above should be interpreted as illustrative in all aspects and not restrictive.

What is claimed is:

1. A method of establishing a tinnitus animal model, comprising:
(S1) exposing animals to restraint stress (the animals are not humans); and
(S2) determining whether the restraint stress-exposed animals are tinnitus-positive,
wherein the restraint stress satisfies any one or more conditions selected from the group consisting of:
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

2. The method of claim 1, wherein the restraint stress is characterized by complete restraint, soft restraint, repeated restraint, and open restraint conditions.

3. The method of claim 1, wherein, in (S1), the restraint stress is applied for 30 to 180 minutes for 5 to 13 days.

4. The method of claim 1, wherein, in (S2), the determining of whether the animals are tinnitus-positive is performed by determining the animals as tinnitus-positive when the mean gap/no-gap (GN) ratio calculated by the following formula after exposure to restraint stress exceeds the 95% confidence interval of the GN ratio before exposure to restraint stress in a gap pre-pulse inhibition of acoustic startle (GPIAS) analysis method.
[GN ratio]=(mean amplitude of gap-trials/mean amplitude of no-gap-trials)×100

5. The method of claim 1, wherein the animal is any one selected from the group consisting of a mouse and a rat.

6. The method of claim 1, wherein the tinnitus animal maintains hearing function.

7. The method of claim 1, wherein the tinnitus animal has an increased plasma corticosterone level.

8. The method of claim 1, wherein the tinnitus animal shows any one or more selected from the group consisting of the following characteristics in any one or more regions selected from the group consisting of Cornu Ammonis 1 (CA1), Cornu Ammonis 2 (CA2), and Cornu Ammonis 3 (CA3) of the hippocampus; the amygdala, and the inferior colliculus:
a significantly decreased gamma-aminobutyric acid (GABA) expression level, or significantly increased expression level(s) of any one or more selected from the group consisting of NMDA receptor subunit 1 (NR1) and NMDA receptor subunit 2B (N2B).

9. A kit for establishing a tinnitus animal model, comprising a restraint stress-inducing apparatus, and instructions,
wherein the restraint stress satisfies any one or more conditions selected from the group consisting of the following (the animals are not humans):
i) restraint density conditions consisting of complete restraint in which almost no movement is possible, partial restraint in which only movement of the head and torso is possible, and loose restraint in which voluntary movement is impossible but position changes are possible;
ii) restraint pressure conditions consisting of soft restraint applied with a flexible material, hard restraint applied with a rigid material, and variable-pressure restraint with adjustable pressure;
iii) restraint duration conditions consisting of single short-term restraint, repeated restraint lasting less than 14 days, and chronic restraint lasting 14 days or more; and
vi) restraint exposure conditions consisting of enclosed restraint and open restraint.

10. The kit of claim 9, wherein the instructions teach any one of claims 1 to 8.
